(19) United States
(12) Patent Application Publication
(10) Pub. No.: US 2019/0038142 A1
(43) Pub. Date: Feb. 7, 2019

(54) OPTICAL SENSOR DEVICE AND ELECTRIC DEVICE COMPRISING AN OPTICAL SENSOR DEVICE

(71) Applicant: OSRAM Opto Semiconductors GmbH, Regensburg (DE)

(72) Inventor: Christoph Goeltner, Cupertino, CA (US)

(21) Appl. No.: 15/666,922

(22) Filed: Aug. 2, 2017

(51) Int. Cl. A61B 5/0205; A61B 5/024; A61B 5/00

(52) U.S. Cl. CPC A61B 5/0205; A61B 5/0478; A61B 5/681; A61B 5/02438

(57) ABSTRACT

An optical sensor device includes at least one integrated skin electrode. An optical sensor device includes an optical sensor that detects a non-electric biosignal, at least one skin electrode that detects an electric biosignal, and holding means that support the optical sensor and in which the at least one skin electrode is integrated. An electric device adapted for attachment to a human or animal body and including an optical sensor device including at least one integrated skin electrode. A watch including the electric device.

### TECHNICAL FIELD

This disclosure concerns an optical sensor device comprising at least one integrated skin electrode and an electric device comprising such an optical sensor device.

### BACKGROUND

Optical and electrical sensors may be used to detect and monitor biosignals. A biosignal is any signal in a human being or an animal that can, usually continually, be measured and monitored. Usually, biosignals are time-varying signals. In conventional devices, optical and electrical sensors are handled by a set of two completely separate component sets.

There is thus a need to provide a more compact product design for a sensor device.

### SUMMARY

I provide an optical sensor device including at least one integrated skin electrode.

I also provide an optical sensor device including an optical sensor that detects a non-electric biosignal, at least one skin electrode that detects an electric biosignal, and holding means that support the optical sensor and in which the at least one skin electrode is integrated.

I further provide an electric device adapted for attachment to a human or animal body and including an optical sensor device including at least one integrated skin electrode.

I further yet provide a watch including the electric device adapted for attachment to a human or animal body and including an optical sensor device including at least one integrated skin electrode.

### BRIEF DESCRIPTION OF THE DRAWINGS

FIG. 1a shows a cross section of an example of an optical sensor device.

FIG. 1b shows a top view of the example of the optical sensor device shown in FIG. 1a.

FIG. 2 shows a top view of a further example of an optical sensor device.

FIG. 3 shows a top view of a further example of an optical sensor device.

FIG. 4 shows a cross section of an example of an electric device including an optical sensor device.

### REFERENCE NUMERALS

1 sensor device

2 holding means

4 electromagnetic radiation emitter

5 radiation emitting side

6 electromagnetic radiation detector

8 skin electrode

10 contact

14 contact

18 electric device

19 strap

20 wrist

22 bone

24 blood vessel

### DETAILED DESCRIPTION

My optical sensor device comprises at least one integrated skin electrode. The at least one skin electrode is incorporated in the optical sensor in an integrated fashion such that a whole is formed. The optical sensor device may form, e.g. an electric assembly or an electric module. A skin electrode is a small piece of metal or other material that is used to to provide an electric current or an electric voltage to a human or animal body, or to take an electric current or an electric voltage from a human or animal body.

The optical sensor device may further comprise a protective holding means in which the at least one skin electrode is integrated. The protective holding means supports and protects an optical sensor. The protective holding means may be formed as a component package, which is a supporting case for the optical sensor, or as a component frame, holding the optical sensor being attached onto or into the component frame. The skin electrodes are integrated in the component package or frame. Integration also includes that the skin electrodes or at least parts thereof are arranged at the outside of the protective holding means. Combining an optical sensor with an integrated electrode or a set of electrodes allows for a miniaturized sensor device.

Such an optical sensor device allows a much more compact product design in wearables and in the medical space. Wearables, also known as wearable computers, are miniature electronic devices attachable to the human body or animal body. They may be worn under, with or on top of clothing. Wearables may be used in the field of health and prevention, e.g. as blood pressure and pulse monitors. Further examples are fitness wristbands, e.g. to track daily movements and pulse, and watches with extra features.

The optical sensor device may comprise an electromagnetic radiation emitter and an electromagnetic radiation detector that form the optical sensor. The electromagnetic radiation emitter, e.g. a light emitting diode (LED), emits electromagnetic radiation onto the skin of a human being or an animal. The radiation may have one or more, in particular two, wavelengths, or one or more spectral ranges. In any case, it should not harm the human or animal body. The radiation may be light, in particular visible or infrared light. The electromagnetic radiation detector, e.g. a photodetector or a photodiode, detects the radiation reflected by or passed through the human being or the animal. Changes in absorption, which may be caused by pulsing blood flow or varying oxygen saturation, affect the amount of detected radiation.

The electromagnetic radiation emitter and the electromagnetic radiation detector as well as the at least one skin electrode may be arranged on a same radiation emitting side of the optical sensor device. When in use, this radiation emitting side is attached to the skin of the human being or the animal, which allows to the detect biosignals by the skin electrodes as well as the optical sensor.

The device may comprise a contact conductively connected with one skin electrode that provides a potential applied at the skin electrode. Usually one contact per skin electrode is provided. Further contacts are provided to control the optical sensor and provide its measurement signals. The contacts may be arranged on a side located opposite the radiation emitting side. The contacts for the skin electrodes conductively connect to the skin electrodes by through-connection vias or wires.

The sensor device may be suitable to detect non-electric and electric biosignals. The latter may include potentials, electromagnetic fields and currents. They may be measured by the at least one electrode. The non-electric biosignal may be measured by an opto-electric transducer formed by the optical sensor that provides an electrical signal indicating a value of the non-electric biosignal. The sensor device comprises an optical sensor that detects a non-electric biosignal and at least one skin electrode that detects an electric biosignal, preferably potentials on the skin. The measured electric, and optically obtained non-electric biosignals may form the basis of subsequent monitoring and processing. Electrodiagnosis-related measurement methods may be one form of such monitoring and processing. They gather information about medical parameters recording the electrical activity of body parts or by measuring their response to external electrical stimuli. Examples of this are electroencephalography, electrocardiography, electromyography and el ectroplethysmography.

An electric device comprising an optical sensor device including at least one integrated skin electrode is suitable for being attached to a human or animal body. The electric device may be attached by straps or the like. Alternatively, the electric device may be integrated in clothes attaching the sensor device to the skin when worn. The electric device may be suitable for detecting a non-electric biosignal and an electric biosignal to measure body functions of a human being or an animal. Such biosignals may be monitored and processed inside or outside of the electric device. The electric device may be suitable for detecting data for photoplethysmography and/or one of the group containing electroencephalography, electrocardiography, electromyography and electroplethysmography, or performing photoplethysmography and one of the group containing electroencephalography, electrocardiography, electromyography and electroplethysmography. The electric device may be suitable to measure a heart rate of a human being or an animal, which may be an extra function in a watch or a fitness tracker.

Non-limiting examples will now be described with reference to the accompanying drawings.

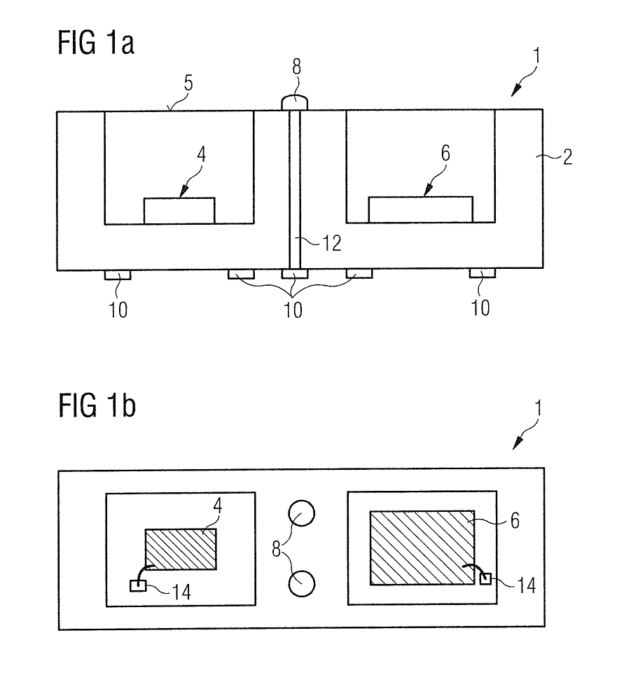

FIG. 1a shows a cross section of an example of an optical sensor device 1. FIG. 1b shows a top view of the example of the optical sensor device 1 shown in FIG. 1a.

The optical sensor device 1 comprises an optical sensor including an electromagnetic radiation emitter 4 and an electromagnetic radiation detector 6 arranged in cavities of a protective holding means 2. The holding means 2 is a frame or package supporting and protecting the electromagnetic radiation emitter 4 and the electromagnetic radiation detector 6. The electromagnetic radiation emitter 4 is suitable for emitting electromagnetic radiation, which may be light, in particular visible or infrared light or a combination thereof In this case, the electromagnetic radiation emitter 4 and detector 6 may be regarded as a light emitter and a light detector, respectively. The spectrum of the electromagnetic radiation may be limited to one or more ranges or one or more wavelengths. The radiation is emitted from a radiation emitting side 5 of the optical sensor device 1, which is a top side shown in FIG. 1a. The electromagnetic radiation emitter 4 may be formed as a light emitting diode, LED. The electromagnetic radiation detector 6, which may be formed as a photodiode, is suitable for detecting electromagnetic radiation and providing an electrical signal indicating the detected radiation. The electromagnetic radiation detector 6 is suitable for detecting equal or similar spectral ranges or wavelengths as emitted by the electromagnetic radiation emitter 4.

The optical sensor device 1 further comprises two skin electrodes 8 arranged on the radiation emitting side 5 of the sensor device 1. The skin electrodes 8 are integrated in the holding means 2 such that the holding means 2 supports them, and at least heads of the skin electrodes 8 are arranged on the surface of the holding means 2 or extend beyond it. The skin electrodes 8 are suitable for being placed onto the skin of a human being or an animal. In this example, the skin electrodes 8 are arranged between the electromagnetic radiation emitter 4 and the electromagnetic radiation detector 6.

The optical sensor device 1 further comprises contacts 10 to apply electrical signals to and provide electrical signals by the optical sensor device 1, thereby controlling it. The contacts 10 allow control of the electromagnetic radiation emitter 4 and the electromagnetic radiation detector 6 as well as the skin electrodes 8. In this example, the contacts 10 are arranged on a side located opposite to the radiation emitting side 5. Some contacts 10 conductively connect to the pair of skin electrodes 8 by through-connection vias or an electrically conductive wire 12. Other contacts 10 conductively connect to contacts 14 or components connected to the electromagnetic radiation emitter 4 and the electromagnetic radiation detector 6. The contacts 10 at the backside of the optical sensor device 1 allow connecting it with an electronic circuitry.

Generally, the core of the sensor device 1 may be considered as an optical sensor device in which skin electrodes 8 are integrated. An electrically conductive wire or via connects each skin electrode head, which is in contact with the skin, to the backside of the sensor device, where it can be connected to an electronic circuitry.

The optical sensor device 1 is suitable for detecting non-electric and electric biosignals. A non-electric biosignal has to be transformed by a transducer into an electric signal that may be stored, displayed or processed. In this example, the optical sensor serves as a photo-electric transducer to detect a non-electric biosignal. An electric biosignal, which may include potentials, electromagnetic fields or currents, may be detected by electrodes before being stored, displayed or processed. The non-electric and electric biosignals which can be measured by the sensor device 1 allow, for example, photoplethysmography based on the non-electric biosignal and, for example, electroencephalography, electrocardiography, electromyography and electroplethysmography, all based on the electric biosignal. Nevertheless, other electrodiagnostic-related measurement techniques and measurement techniques based on the optical signal are possible.

A plethysmography allows measuring changes in volume within an organ or the whole body, usually resulting from fluctuations in the amount of blood or air it contains. Photoplethysmography (PPG) is based on optically obtained signals. Electroplethysmography is based on signals detected by electrodes. Electroencephalography (EEG) is an electrophysiological monitoring method to record electrical activity of the brain. Electrocardiography (ECG) is the process of recording the electrical activity of the heart using electrodes placed on the skin. These electrodes detect electrical changes on the skin that arise from the heart muscle's electrophysiologic pattern of depolarizing and repolarizing during each heartbeat. An electrocardiogram show the detected voltages over time. Electromyography (EMG) is a technique to evaluate and record the electrical activity produced by skeletal muscles.

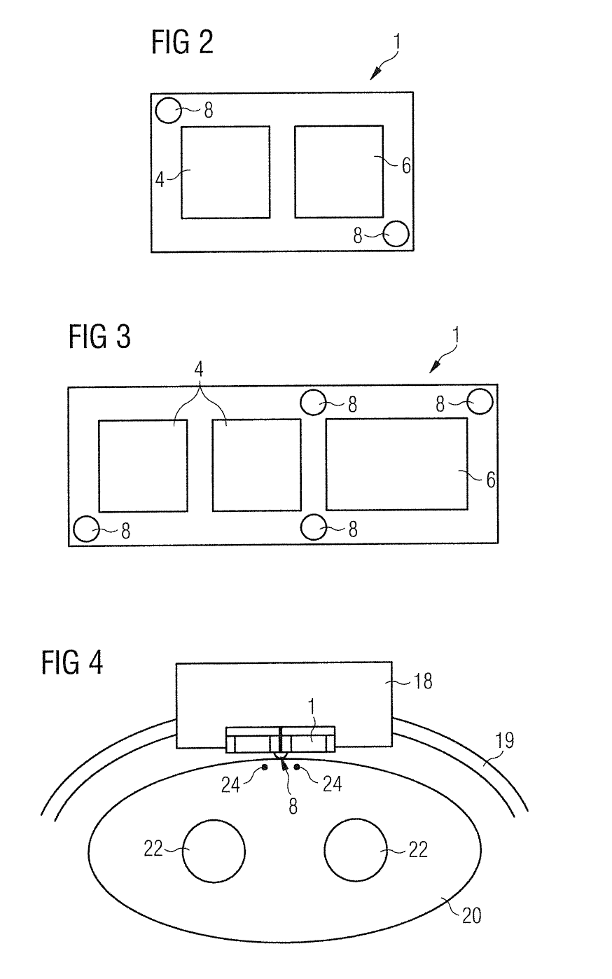

FIG. 2 shows a top view of a further example of an optical sensor device 1. To avoid a mere repetition, only the differences with respect to FIGS. 1a and 1b are described.

In FIG. 2, the skin electrodes 8 are arranged at the outer edges of the optical sensor device 1, the skin electrodes 8 being located diagonally opposite of each other.

FIG. 3 shows a top view of a further example of an optical sensor device. To avoid a mere repetition, only the differences with respect to FIGS. 1a and 1b are described.

In FIG. 3, two electromagnetic radiation emitters 4 are provided. The sensor device 1 comprises four skin electrodes 8. The skin electrodes 8 are arranged between the electromagnetic radiation emitters 4 and the electromagnetic radiation detector 6 as well as at the outer edges, diagonally opposite of each other. This example illustrates that any number and arrangement of skin electrodes 8, electromagnetic radiation emitters 4 and electromagnetic radiation detectors 6 can be implemented, depending on the overall geometry of the optical sensor device 1 and the need for number and spacing of the skin electrodes 8.

FIG. 4 shows a cross section of an example of an electric device 18 comprising an optical sensor device 1. The latter has been exemplarily described in connection with FIGS. 1a, 1b, 2 and 3. The sensor device 1 is arranged such that the radiation emitting side is placed at an outside of the electric device 18, the outside facing towards a human skin or an animal skin when worn by a human being or an animal, respectively.

The electric device 18 is a wrist-worn device 18, e.g. a watch. The wrist-worn device 18 includes a strap 19 to fasten it to the wrist 20 including bones 22 and blood vessels 24.

The electric device 18 connects to the sensor device 1 by the contacts 10. The electric device 18 controls the skin electrodes 8 and the optical sensor including the electromagnetic radiation emitter 4 and the electromagnetic radiation detector 6. The skin electrodes 8 detect time-dependent potential changes on the skin and provide these signals at the contacts 10, which are connected to the skin electrodes 8. In electrocardiography (ECG), potential changes arising during each heartbeat are considered. The detected signals allow to determine, e.g. the pulse frequency. Nevertheless, the skin electrodes may also be used to provide signals for other measurements.

Furthermore, the electric device 18 controls the optical sensor. Signals that control the electromagnetic radiation emitter 4 are applied to some of the contacts 10. Signals indicating the detected radiation by the electromagnetic radiation detector 6 are provided at some other contacts 10.

The electromagnetic radiation emitter 4 emits the electromagnetic radiation onto the skin, which absorbs part of the electromagnetic radiation and reflects part of the electromagnetic radiation. The electromagnetic radiation detector 6 measures changes in electromagnetic radiation absorption or reflection, which may be caused by perfusion of blood to the dermis and subcutaneous tissue of the skin. The detected signal may form the basis of a photoplethysmography (PPG). With each cardiac cycle the heart pumps blood to the periphery. Thus, a pressure pulse can detected. Moreover, photoplethysmography allows monitoring oxygen saturation. Because blood flow to the skin can be modulated by a number of other physiological systems, the PPG can also be used to monitor breathing, hypovolemia, and other circulatory conditions.

The electric device 18 may be suitable for displaying and/or processing the measured signals. It may determine, display and monitor the heart frequency and other parameters. This example shows how the sensor device 1 can be used in a wrist-worn device 18 to optically sense the heart rate and sense the electrical signal to determine an electrocardiogram.

The scope of protection is not limited to the examples given herein above. This disclosure is embodied in each novel characteristic and each combination of characteristics, which particularly includes every combination of any features stated in the appended claims, even if the feature or combination of features is not explicitly stated in the claims or examples.

1. An optical sensor device comprising at least one integrated skin electrode.

2. The optical sensor device according to claim 1, further comprising a holding means in which the at least one skin electrode is integrated.

3. The optical sensor device according to claim 2, wherein the holding means is formed as a component package supporting an optical sensor or as a component frame supporting an optical sensor.

4. The optical sensor device according to claim 1, comprising an electromagnetic radiation emitter and an electromagnetic radiation detector.

5. The optical sensor device according to claim 4, having a radiation emitting side, wherein the electromagnetic radiation emitter, the electromagnetic radiation detector and the at least one skin electrode are arranged on the radiation emitting side.

6. The optical sensor device according to claim 1, further comprising at least one contact conductively connected to the at least one skin electrode.

7. The optical sensor device according to claim 6, wherein the at least one contact is arranged on a side located opposite the radiation emitting side, the at least one contact conductively connected to the at least one skin electrode by a through-connection via or a wire.

8. The optical sensor device according to claim 1, adapted to detect a non-electric biosignal and an electric biosignal.

9. An optical sensor device comprising an optical sensor that detects a non-electric biosignal, at least one skin electrode that detects an electric biosignal, and holding means that support the optical sensor and in which the at least one skin electrode is integrated.

10. An electric device adapted for attachment to a human or animal body and comprising an optical sensor device comprising at least one integrated skin electrode.

11. The electric device according to claim 10, adapted to detect a non-electric biosignal and an electric biosignal to measure body functions of a human being or an animal.

12. The electric device according to claim 10, adapted to measure data for photoplethysmography and one of the group containing electroencephalography, electrocardiography, electromyography and electroplethysmography and/or for performing photoplethysmography and one of the group containing electroencephalography, electrocardiography, electromyography and el ectroplethysmography.

13. The electric device according to claim 10, adapted to measure a heart rate of a human being or an animal.

14. A watch comprising the electric device according to claim 10.
